(19) United States
(12) Patent Application Publication
(10) Pub. No.: US 2009/0269800 A1
(43) Pub. Date: Oct. 29, 2009

(54) DEVICE AND METHOD FOR PROCESSING CELL SAMPLES

(76) Inventors: Todd COVEY, San Carlos, CA (US); Garry P. Nolan, San Francisco, CA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/432,239

(22) Filed: Apr. 29, 2009

Related U.S. Application Data
(60) Provisional application No. 61/048,657, filed on Apr. 29, 2008.

(51) Int. Cl. C12Q 1/02; C12M 1/00

(52) U.S. Cl. 435/29; 435/309.1; 435/287.2; 435/287.1

(57) ABSTRACT

A cell analysis device and method is described which enables the user to efficiently treat cells in a sample. The cells can be treated with reagents at a time and place proximate to collection and may be handled in a automated or semi automated fashion.

### PRIORITY

This application claims priority to provisional application No. 61/048,657, filed Apr. 29, 2008, the disclosure of which is hereby incorporated by reference in its entirety for all purposes.

### BACKGROUND OF THE INVENTION

### Field of the Invention

The present invention relates to the processing of biological samples. More specifically, the present invention relates to the processing of fluid samples and subsequent analysis.

### SUMMARY OF THE INVENTION

One embodiment of the invention is a system for preparing cells for analysis, comprising at least one cartridge containing a plurality of chambers, a manifold fluidly connected to the plurality of chambers, at least one fluid line to deliver the plurality of cells to the chambers; and a plurality of reagents fluidly connected to a plurality of chambers. In one embodiment the reagent is a modulator and is present in the plurality of chambers prior to, or after the addition of the plurality of cells.

Another embodiment of the invention is a method for preparing cells for flow cytometry analysis, comprising obtaining a plurality of cells from an individual, directing the cells through a manifold into a plurality of chambers, contacting the cells in the chambers with one or more modulators, optionally contacting the cells with a fixative; and analyzing the cells using a flow cytometer.

The method may also specify contacting the cells with a stain in the chambers and detecting a stain on a plurality of cells using the flow cytometer. The cells may be present in whole blood and the chamber holding the whole blood may have a vent. The method may also comprise drawing the cells into the chambers using a vacuum. The method may also comprise injecting the cells into the chamber with pressure. The method may also comprise allowing gravimetric or centrifugal force to inject cells into the chamber. The volume of any chamber can be between about 0.1 microliters and about 10 mls, or between 0.1 mls and 10 mls or between 0.1 microliters and 10 microliters, and different chambers may have different volumes. In various embodiments, the chambers are formed into a structure called a cartridge. Some embodiments of the cartridge can have chambers which are closed end tubes that are held together in a framework, they can be in a microtiter plate, or in a lab-on-a-chip. The microtiter plate may have 94, 384 or 1536 wells.

In another embodiment, the chamber has an associated identifier, such as a bar code, RFID, or DNA bar code and can be agitated using bubbles or physical agitation. Also, a filter can be added between the whole blood and chamber to concentrate certain cells, such as white blood cells, or remove cells or impurities.

In another embodiment the modulator can be added with an attachable device which is fluidly connected to a plurality of chambers and can be designed for flexibly adding particular modulators to particular chambers.

In another embodiment, the at least one cartridge for preparing cells for flow cytometry comprises a housing including a plurality of fluid chambers constructed and arranged to retain fluid containing cells, said housing including an identifier such as a bar code, the housing is fluidly connected to a source containing cells, a manifold fluidly connected to the housing, cell stimulating reagents, and time release cell fixing buffers.

In another embodiment, the method encompasses collecting the cells directly from a venous puncture of the patient.

In another embodiment, the method encompasses collecting the cells first in one device (a CPT tube for example), and then using a second needle (or a needle with an air flow allowance) to remove the cells from that tube and place through the manifold into the device described herein (a series of chambers or tubes).

In another embodiment the invention is a cell analysis system comprising a fluid container comprising a plurality of cells, a plurality of chambers, a fluid delivery line to connect the fluid container to the plurality of chambers, an automated device for programmed delivery of liquid or solid reagents to the plurality of chambers, and a flow cytometer. An automated syringe can be used to deliver reagents and a computer system may be used for controlling fluid flow into a number of chambers comprising software stored on storage medium.

One embodiment of the present invention involves a seal for each junction between the lines, manifolds and chambers to retain the fluid within the chamber. By forming a sealed chamber in which sample fluids and solid or liquid reagents can easily be introduced, a practical device is provided to prepare multiple samples for flow cytometry at a time that is close to when a sample is taken from an individual.

In other embodiments, the body is formed by joining multiple pieces together, for example by injection molding pieces for assembly. The concept of assembling the body from multiple pieces is advantageous. For example, the various features of the cartridge or block are formed without requiring complex machining or designing. Thus, the cartridges are produced at a relatively low cost.

In connection with one aspect of the invention, a method for making the cell cartridge is disclosed. In particular, the method comprises the steps of first forming a cartridge with a plurality of chambers. Then a cartridge can be mated to a package having a reaction chamber with fluid inlets. When mated, the cartridge is in fluid communication with the reaction chamber and the reagents may be brought in contact with the cell sample.

### BRIEF DESCRIPTION OF THE DRAWINGS

FIG. 1 shows one embodiment of the invention in which a plurality of samples are taken and distributed to a plurality of chambers via a manifold.

FIG. 2 shows another embodiment of the invention in which a plurality of samples are taken and distributed to a plurality of chambers via a manifold and a cassette-like attachment may be used for the reagents.

FIG. 3 shows a diagram of an example of one embodiment in which flow cytometry preparation assay is used.

FIG. 4 shows one example of how the sample may be taken and processed with reagents.

FIG. 5 describes some elements of the system shown in FIG. 4.

FIG. 6 shows an alternative embodiment of the system.

FIG. 7 shows another alternative embodiment of the present invention.

FIG. 8 shows another alternative embodiment of the present invention.

### DESCRIPTION OF THE SPECIFIC EMBODIMENTS

### General

One embodiment of the present invention is a device or cartridge to house at least one fluid sample, and preferably a plurality of fluid samples containing biological cells to be investigated upon the addition of reagents. Preferably, the device can remove a sample from a larger volume of whole blood and move the sample to a physical structure housing one or more chambers. The chambers may have the appropriate reagents within the chamber prior to the addition of the sample or the reagents may be added thereafter. Preferably, the samples are added in an efficient manner, such as by single delivery for all reagents to any one of a multiple set of chambers.

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including, but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

One aspect of the present invention is a device that is used to prepare samples for flow cytometry. Samples may be derived from various bodily tissues or sources and one embodiment, such as flow cytometry, can be practiced on samples derived from whole blood. Once the blood is taken, it may be held or stored in a container, such as a tube. See Lony, C. L, et. al., Clin Diagn Lab Immunol. 1998 May; 5(3): 392-398. Thereafter, smaller aliquots of the sample may be treated with reagents prior to the cytometry process. For example, one embodiment of the present invention treats cells that are present in the sample with one or more modulators or potentiators to initiate a cellular response. The present invention incorporates information disclosed in other applications and texts that are mentioned throughout this disclosure. For example, the following patent and other publications are hereby incorporated by reference in their entireties: Haskell et al, Cancer Treatment, 5^{th} Ed., W.B. Saunders and Co., 2001; Alberts et al., The Cell, 4^{th} Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Immunobiology, Janeway et al. 7^{th} Ed., Garland; Weinberg, The Biology of Cancer, 2007; and Leroith and Bondy, Growth Factors and Cytokines in Health and Disease, A Multi Volume Treatise, Volumes 1A and 1B, Growth Factors, 1996. Patents and patent applications that are also incorporated by reference include U.S. Pat. Nos. 7,393,656 and 7,381,535 and U.S. Ser. Nos. 10/193,462; 11/655,785; 11/655,789; 11/655,821; and 11/338,957. See also U.S. Pat. Nos. 7,326,577, 5,422,277, 5,122,453 and 5,597,688, and U.S. Pub. No. 2006/0141549. Relevant articles include High-content single-cell drug screening with phosphospecific flow cytometry, Krutzik et. Al., Nature Chemical Biology, 23 Dec. 2007; Irish et. al., Flt3 Y591 duplication and Bcl-2 over expression are detected in acute myeloid leukemia cells with high levels of phosphorylated wild-type p53, Neoplasia, 2007, Irish et. al., Single cell profiling of potentiated phospho-protein networks in cancer cells, Cell, Vol. 118, 1-20 Jul. 23, 2004; Krutzik et al, J Immunol 2005, 175:2357-235; Schulz et al., Current Protocols in Immunology, 2007, 78:8.17.1-20; Chow S, et al., Measurement of the MAP kinase activation by flow cytometry using phospho-specific antibodies to MEK and ERK: potential for pharmacodynamic monitoring of signal transduction inhibitors. Cytometry 2001; 46:72-78; Perez, 0 D & Nolan, G P, Simultaneous measurement of multiple active kinase states using polychromatic flow cytometry, Nat. Biotechnol 2002; 20; 1551-162; Jacobberger, J W, Flow Cytometric Analysis of Intracellular Protein Epitopes. Immunophenotyping 2000; 361-409; Pizzolo, G, et al. Detection of membrane and intracellular antigens by flow cytometry following ORTHO PermeaFix fixation, Leukemia. 1994 April; 8(4):672-6; Francis, C & Connelly, M C, Rapid single-step method for flow cytometric detection of surface and intracellular antigens using whole blood, Cytometry, 1996 Sep. 1; 25(1):58-70; Baatout, S & Cheta, N, Permeafix: a useful tool to detect antigens and DNA in flow cytometry, Rom J Intern Med. 1997 Jan.-December; 35(1-4):133-5; Murray, M, et al., ORTHO Permeafix fixation is not suitable for the flow cytometric detection of nuclear terminal transferase in acute myloid leukemia cells. Leukemia. 1995 January; 9(1):226-8; and Metso, T, et al., Identification of intracellular markers in induced sputum and bronchoalveolar lavage samples in patients with respiratory disorders and healthy persons. Respir Med. 2002 November; 96(11):918-26. Generally, the discussion outlined in U.S. Pat. No. 7,393,656 includes a general description of a preferred process involving Activation; Types of Activation; Potentiation; Detection of State; Binding Elements; Labels; Alternative Activation State Indicators; FACS Analysis; Additional Techniques; Types of Bioactive Candidates that Can be Used; General Screening Methods; Screening of Agents in the Potentiated Model; Analysis; and Hardware/General Techniques. '656 also provides substantial detail on examples of the process.

In addition to the references cited above, the practice of the present invention may employ, unless otherwise indicated, conventional techniques and descriptions of flow cytometry, organic chemistry, polymer technology, molecular biology (including recombinant techniques), cell biology, cancer biology, biochemistry, and immunology, which are within the skill of the art. Such conventional techniques can be found in articles, patents, commercial websites, as well as other sources as referenced above. Other conventional techniques can be shown in standard laboratory manuals such as those recited above and also including Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach” 1984, IRL Press, London, Nelson and Cox (2000), Lehninger, Principles of Biochemistry 3rd Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention also contemplates the use of a computer which may operate various instrumentation, liquid handling equipment or analysis steps of the invention. For instance, a computer controlled collection, handling, or analysis system may be used to control, activate, initiate, continue or terminate any step or process of the invention as herein described. In one embodiment, a computer device may be used to control, activate, initiate, continue or terminate the obtaining of cells from a subject, the handling and/or movement of cells or fluids or reagents into and through the system or device as herein described, the deposition of cells into one or more chambers or plurality of chambers in one or more cartridges, the handling or movement of one or more reagents to one or more chambers or plurality of chambers in one or more cartridges, the obtaining or analysis of data, etc.

The computer may be any type of computer platform such as a workstation, a personal computer, a server, or any other present or future computer. The computer typically includes known components such as a processor, an operating system, system memory, memory storage devices, and input-output controllers, input-output devices, and display devices. Display devices may include display devices that provides visual information, this information typically may be logically and/or physically organized as an array of pixels. A Graphical the user interface (GUI) controller may also be included that may comprise any of a variety of known or future software programs for providing graphical input and output interfaces such as for instance GUI'S. For example, GUI's may provide one or more graphical representations to a the user, and also be enabled to process the user inputs via GUI's using means of selection or input known to those of ordinary skill in the related art.

It will be understood by those of ordinary skill in the relevant art that there are many possible configurations of the components of a computer and that some components that may typically be included in a computer are not shown, such as cache memory, a data backup unit, and many other devices. The processor may be a commercially available processor such as an Itanium® or Pentium® processor made by Intel Corporation, a SPARC® processor made by Sun Microsystems, an Athalon™ or Opteron™ processor made by AMD corporation, or it may be one of other processors that are or will become available. Some embodiments of the processor may also include what are referred to as Multi-core processors and/or be enabled to employ parallel processing technology in a single or multi-core configuration. For example, a multi-core architecture typically comprises two or more processor “execution cores”. In the present example each execution core may perform as an independent processor that enables parallel execution of multiple threads. In addition, those of ordinary skill in the related will appreciate that the processor may be configured in what is generally referred to as 32 or 64 bit architectures, or other architectural configurations now known or that may be developed in the future.

The processor executes operating system, which may be, for example, a Windows®-type operating system (such as Windows® XP) from the Microsoft Corporation; the Mac OS X operating system from Apple Computer Corp. (such as 7.5 Mac OS X v10.4 “Tiger” or 7.6 Mac OS X v10.5 “Leopard” operating systems); a Unix® or Linux-type operating system available from many vendors or what is referred to as an open source; another or a future operating system; or some combination thereof. The operating system interfaces with firmware and hardware in a well-known manner, and facilitates processor in coordinating and executing the functions of various computer programs that may be written in a variety of programming languages. The operating system typically in cooperation with the processor, coordinates and executes functions of the other components of computer. The operating system also provides scheduling, input-output control, file and data management, memory management, and communication control and related services, all in accordance with known techniques.

The system memory may be any of a variety of known or future memory storage devices. Examples include any commonly available random access memory (RAM), magnetic medium such as a resident hard disk or tape, an optical medium such as a read and write compact disc, or other memory storage device. Memory storage devices may be any of a variety of known or future devices, including a compact disk drive, a tape drive, a removable hard disk drive, USB or flash drive, or a diskette drive. Such types of memory storage devices typically read from, and/or write to, a program storage medium (not shown) such as, respectively, a compact disk, magnetic tape, removable hard disk, USB or flash drive, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with memory storage device.

In some embodiments, a computer program product is described comprising a computer usable medium having control logic (computer software program, including program code) stored therein. The control logic, when executed by a processor, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers could include any of a variety of known devices for accepting and processing information from a user, whether a human or a machine, whether local or remote. Such devices include, for example, modern cards, wireless cards, network interface cards, sound cards, or other types of controllers for any of a variety of known input devices. Output controllers of input-output controllers could include controllers for any of a variety of known display devices for presenting information to a user, whether a human or a machine, whether local or remote. In the illustrated embodiment, the functional elements of computer communicate with each other via system bus. Some of these communications may be accomplished in alternative embodiments using network or other types of remote communications.

As will be evident to those skilled in the relevant art, an instrument control and image processing application, such as for instance an implementation of instrument control and image processing applications, if implemented in software, may be loaded into and executed from system memory and/or memory storage device. All or portions of the instrument control and image processing applications may also reside in a read-only memory or similar device of memory storage device, such devices not requiring that the instrument control and image processing applications first be loaded through input-output controllers. It will be understood by those skilled in the relevant art that the instrument control and image processing applications, or portions of it, may be loaded by processor in a known manner into system memory, or cache memory (not shown), or both, as advantageous for execution. Library files, calibration data, experiment data, and internet client data can be stored in system memory. For example, experiment data could include data related to one or more experiments or assays such as excitation wavelength ranges, emission wavelength ranges, extinction coefficients and/or associated excitation power level values, or other values associated with one or more fluorescent labels. Additionally, internet client may include an application enabled to accesses a remote service on another computer using a network that may for instance comprise what are generally referred to as “Web Browsers”. In the present example some commonly employed web browsers include Microsoft® Internet Explorer 6 with SPI available from Microsoft Corporation, Mozilla Firefox® 1.5 from the Mozilla Corporation, Safari 2.0 from Apple Computer Corp., or other type of web browser currently known in the art or to be developed in the future. Also, in the same or other embodiments the internet client may include, or could be an element of, specialized software applications enabled to access remote information via a network such as network.

The network may include one or more of the many various types of networks well known to those of ordinary skill in the art. For example, the network may include a local or wide area network that employs what is commonly referred to as a TCP/IP protocol suite to communicate, that may include a network comprising a worldwide system of interconnected computer networks that is commonly referred to as the internet, or could also include various intranet architectures. Those of ordinary skill in the related arts will also appreciate that some the users in networked environments may prefer to employ what are generally referred to as “firewalls” (also sometimes referred to as Packet Filters, or Border Protection Devices) to control information traffic to and from hardware and/or software systems. For example, firewalls may comprise hardware or software elements or some combination thereof and are typically designed to enforce security policies put in place by the users, such as for instance network administrators, etc.

Instrument control and image processing applications may comprise any of a variety of known or future image processing applications. Typically, embodiments of applications may be loaded into system memory and/or memory storage device.

Those of ordinary skill in the related art will appreciate that applications may be stored for execution on any compatible computer system, such as computer. Embodiments of applications may advantageously provide what is referred to as a modular interface for one or more computers or workstations and one or more servers, as well as one or more instruments. The term “modular” as used herein generally refers to elements that may be integrated to and interact with a core element in order to provide a flexible, updateable, and customizable platform. For example, as will be described in greater detail below applications may comprise a “core” software element enabled to communicate and perform primary functions necessary for any instrument control and image processing application. Such primary functionality may include communication over various network architectures, or data processing functions such as processing raw intensity data into a .dat file. In the present example, modular software elements, such as for instance what may be referred to as a plug-in module, may be interfaced with the core software element to perform more specific or secondary functions, such as for instance functions that are specific to particular instruments. In particular, the specific or secondary functions may include functions customizable for particular applications desired by the user. Further, integrated modules and the core software element are considered to be a single software application, and referred to as applications.

In one embodiment of the presently described implementation, applications may communicate with, and receive instruction or information from, or control one or more elements or processes of one or more servers, one or more workstations, and one or more instruments. Also, embodiments of server or computer with an implementation of applications stored thereon could be located locally or remotely and communicate with one or more additional servers and/or one or more other computers/workstations or instruments.

In some embodiments, applications may be capable of data encryption/decryption functionality. For example, it may be desirable to encrypt data, files, information associated with GUI's or other information that may be transferred over network to one or more remote computers or servers for data security and confidentiality purposes. For example, some embodiments of probe array may be employed for diagnostic purposes where the data may be associated with a patient and/or a diagnosis of a disease or medical condition. It is desirable in many applications to protect the data using encryption for confidentiality of patient information. In addition, one-way encryption technologies may be employed in situations where access should be limited to only selected parties such as a patient and their physician. In the present example, only the selected parties have the key to decrypt or associate the data with the patient. In some applications, the one-way encrypted data may be stored in one or more public databases or repositories where even the curator of the database or repository would be unable to associate the data with the user or otherwise decrypt the information. The described encryption functionality may also have utility in clinical trial applications where it may be desirable to isolate one or more data elements from each other for the purpose of confidentiality and/or removal of experimental biases.

Various embodiments of applications may provide one or more interactive graphical the user interfaces that allows the user to make selections based upon information presented in an embodiment of GUI. Those of ordinary skill will recognize that embodiments of GUI may be coded in various language formats such as an HTML, XHTML, XML, javascript, Jscript, or other language known to those of ordinary skill in the art used for the creation or enhancement of “Web Pages” viewable and compatible with internet client. For example, the internet client may include various internet browsers such as Microsoft Internet Explorer, Mozilla Firefox, Apple Safari, or other browsers known in the art. Applications of GUI's viewable via one or more browsers may allow the user complete remote access to data, management, and registration functions without any other specialized software elements. The applications may provide one or more implementations of interactive GUI's that allow the user to select from a variety of options including data selection, experiment parameters, calibration values, and probe array information within the access to data, management, and registration functions.

In some embodiments, the applications may be capable of running on operating systems in a non-English format, where applications can accept input from the user in various non-English language formats such as Chinese, French, Spanish etc., and output information to the user in the same or other desired language output. For example, applications may present information to the user in various implementations of GUI in a language output desired by the user, and similarly receive input from the user in the desired language. In the present example, the applications are internationalized such that it is capable of interpreting the input from the user in the desired language where the input is acceptable input with respect to the functions and capabilities of the applications.

Embodiments of the applications also include instrument control features, where the control functions of individual types or specific instruments such as the flow cytometer, an autoloader, or fluid handling system may be organized as plug-in type modules to the applications. For example, each plug-in module may be a separate component and may provide definition of the instrument control features to the applications. As described above, each plug-in module is functionally integrated with the applications when stored in system memory and thus reference to the applications includes any integrated plug-in modules. In the present example, each instrument may have one or more associated embodiments of plug-in module that for instance may be specific to model of instrument, revision of instrument firmware or scripts, number and/or configuration of instrument embodiment, etc. Further, multiple embodiments of plug-in module for the same instrument, such as the flow cytometer may be stored in system memory for use by the applications, where the user may select the desired embodiment of module to employ, or alternatively such a selection of module may be defined by data encoded directly in a machine readable identifier or indirectly via the array file, library files, experiments files and so on.

The instrument control features may include the control of one or more elements of one or more instruments that could, for instance, include elements of a fluid processing instrument, autoloader, or the flow cytometer. The instrument control features may also be capable of receiving information from the one more instruments that could include experiment or instrument status, process steps, or other relevant information. The instrument control features could, for example, be under the control of or an element of the interface of the applications. In some embodiments, a user may input desired control commands and/or receive the instrument control information via one of GUI's. Additional examples of instrument control via a GUI or other interface is provided in U.S. patent application Ser. No. 10/764,663, titled “System, Method and Computer Software Product for Instrument Control, Data Acquisition, Analysis, Management and Storage”, filed Jan. 26, 2004, which is hereby incorporated by reference herein in its entirety for all purposes.

Generally it is desirable to consolidate elements of data or metadata related to an experiment, the user, or some combination thereof, to a single file that is not duplicated where duplication may sometimes be a source of error. The term “metadata” as used herein generally refers to data about data. It may also be desirable in some embodiments to restrict or prohibit the ability to overwrite data in the file. Preferentially, new information may be appended to the file rather than deleting or overwriting information, providing the benefit of traceability and data integrity (i.e. as may be required by some regulatory agencies). For example, a file may be associated with one or more experiments and machine readable identifiers, including bar codes, RFID, dye associated bar codes, elemental bar codes, DNA bar codes, for example see U.S. Pat. No. 5,451,505 which is hereby incorporated by reference in its entirety.

Also continuing the example above, some embodiments of machine readable identifiers, such as barcodes or RFID tags may be capable of “data logging” functionality where, for instance, each RFID tag or label may actively measure and record parameters of interest. In the present example, such parameters of interest may include environmental conditions such as temperature and/or humidity that the implementation may have been exposed to. In the present example, the user may be interested in the environmental conditions because the biological integrity of some embodiments may be affected by exposure to fluctuations of the environment. In some embodiments, the applications may extract the recorded environmental information from the RFID tag or label and store it in the file. In the same or alternative embodiments, the applications may monitor the environmental conditions in real time, where the applications may regularly monitor information provided by one or more RFID tags simultaneously. The applications may further analyze and employ such information for quality control purposes, for data normalization, or other purposes known in the related art. Some examples of RFID embodiments capable to recording environmental parameters include the ThermAssureRF™ RFID sensor available from Evidencia LLP of Memphis Tenn., or the Tempsens™ RFID data logging label available from Exago Pty Ltd. of Australia.

It is understood by the skilled artisan that the steps of the assays provided herein can vary in order. It is also understood, however, that while various options (of compounds, properties selected or order of steps) are provided herein, the options are also each provided individually, and can each be individually segregated from the other options provided herein. Moreover, steps that are known in the art that will increase the sensitivity of the assay are intended to be within the scope of this invention. For example, there may be additionally washing steps, blocking steps, etc.

In one embodiment of the present invention, cells are taken from an individual, for example by a blood draw. Procedures for drawing blood are commonly known and the blood can be collected in various receptacles, such as tubes. Blood drawing tubes are commonly known in the art. Once blood is collected, in one embodiment of the invention, it is prepared and tested. One embodiment uses a flow cytometer. The preparation of the cells involves multiple steps. For example, cells may be tested directly for the presence of a cell surface molecule or marker with, for example, an antibody directed to the specific cell surface molecule or marker. The antibody may be directly conjugated with a detectable marker, such as, for example, a fluorochrome or fluorescent tag which can be detected with a flow cytometer. Alternatively, a secondary antibody may be used to detect the presence of the binding of the first specific antibody. This secondary antibody may be conjugated to a detectable marker or tag. In this instance, the cells may be fixed after detection with the antibody directed to the cell surface molecule with a fixative. Alternatively, the cells may be permeablized and an intracellular molecule may be detected in the same manner. These steps require multiple reagents at various times in the process and it is preferable to add the reagents soon after the blood drawing. Preferably, the reagents are added within 0, 3, 5, 10, 15, 20, 30, 45, or 50 minutes or 1, 2, 3, 4, 5, 6, 7, 8, 9, 10, 12, 15, 24, 30, or 36 hours after collection. In one embodiment, the cells are collected directly by venous puncture and treated with the reagents.

The blood may be treated without processing (whole blood) or may be processed in some fashion. For example, subsets of cells within the blood may be filtered and removed for further use and analysis using standard collection or filtering devices. For example, red blood cells may be filtered out of a sample or specific populations of white blood cells may be filtered out for further analysis. Samples may be whole blood, cell suspensions, cells in a buffy coat sample, or fixed to a solid substrate, such as a bead or plate. Some of these cell concentration devices or techniques may be employed by insertion between the blood collection tube and the chambers. For example, in one embodiment of the present invention a filtering device that separates cells may be placed in a fluid line between the blood collection tube and an element of the present invention. Such devices may include micro card or lab-on-a-chip devices that separate specific subsets of cells from the whole blood. See the references below for appropriate examples of these devices.

Whole blood can also be applied to filters that are engineered to contain pore sizes that select for the desired cell type or class. For example, cells can be filtered out of diluted, whole blood following the lysis of red blood cells by using filters with pore sizes between 5 to 10 μm, as disclosed in U.S. patent application Ser. No. 09/790,673.

After collection of the cells, the cell sample may be removed from the initial tube and placed into chambers for processing through a fluid connection between the tube and the chambers of the present invention. The fluid connection may be any one of a manner of conduits (such as commercial lab tubing) of any appropriate size and shape. Tubing is commercially available through a variety of medical and research suppliers. See the commercially available equipment from Corning, Becton-Dickinson, Sastedt, and Tygon.

One embodiment of the present invention is a device to prepare cells for flow cytometry. Advantages of some embodiments of the present invention include the preparation of multiple cell samples at the same time (higher throughput) and at a time that can be closer to when the cells are removed from an individual. Further advantages of the present invention include consistency of cell preparation and results therefrom, ease of use, and scalability.

In one preferred embodiment of the present invention, the methods of the invention include the use of liquid handling components. The liquid handling systems can include robotic systems comprising any number of components. In addition, any or all of the steps outlined herein may be automated; thus, for example, the systems may be completely or partially automated.

Example instruments that may be useful in the present invention include automated liquid handling instruments such as Sciclone series i1000 or ALH3000 and its Autostacker, or the Zepher from Caliper, the Biomek series, including the NX^{P}, NX, FX and the FX^{P} from Beckman Coulter, including the Bar Code Reading ALP, Stacker Carousel plate hotel, Cytomat conveyor ALP, Automated Tube Bar Code Reader (once microplates are formatted), or Handheld Bar Code Reader.

As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In a preferred embodiment, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In one embodiment of the present invention, platforms for multi-well plates, multi-tubes, holders, cartridges, minitubes, deep-well plates, microfuge tubes, cryovials, square well plates, filters, chips, beads, and other solid-phase matrices or platforms with various volumes are accommodated on an upgradable modular platform for additional processing. This modular platform includes a variable speed orbital shaker, and multi-position work decks for source samples, sample and reagent dilution, assay plates, sample and reagent reservoirs, pipette tips, and an active wash station.

In another embodiment, thermoregulating systems are used for stabilizing the temperature of heat exchangers such as controlled blocks or platforms to provide accurate temperature control of incubating samples from 0° C. to 100° C.

The flexible hardware and software allow instrument adaptability for multiple applications. The software program modules allow creation, modification, and running of methods. The system diagnostic modules allow instrument alignment, correct connections, and motor operations. The customized tools, labware, and liquid, particle, cell and organism transfer patterns allow different applications to be performed. The database allows method and parameter storage. Robotic and computer interfaces allow communication between instruments.

In a preferred embodiment, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory.

These robotic fluid handling systems can manipulate any number of different reagents, including modulators, buffers, fixatives, stains, permeabilizing reagents, samples, washes, assay components such as label probes, etc.

As used herein, the term “cartridge” describes the assembly of chambers which will house the sample for reagent treatment. There are several embodiments of a cartridge in the present invention which are described below. They may range in size and sample volume and may be integrated with other devices.

One embodiment of the invention allows a user to take and partially process a cell sample, like a blood sample, at the point of sampling, like a blood draw station, hospital, doctor's office or lab. The user can fluidly connect the device of the present invention to the whole blood collection device, tube or other container, or the collection device may be integrated into a device of the invention, and distribute it to the multiple chambers thereby placing the cell sample in contact with the reagents to initiate the experimental process soon after the blood draw. At a fixed time, a buffer fixative may be added to stop the biological processes (ie, phosphorylation) and preserve the sample. Thereafter, the cell sample may be sent to a lab which handles the subsequent analysis, such as by flow cytometry analysis, for more processing, which may be at a different location. One embodiment of the present invention involves communication over the internet using a network to provide details about such things like sample collection information, cell or patient phenotypes, treatment regimes, or any other data that may be useful to integrate into the data analysis. The data may be inputted at the point of care or blood collection. The present invention provides faster process at the point of care, easier processing, more uniform handling and consistency, and higher through-put. Not all of the reagents need to be added at the point of sampling. Other reagents may be added later, at another location, such as where the cytometry will be conducted. The samples may be shipped in a state that will ensure their stability, such as in a frozen or chemically static state. The samples may be held at less than −20, −30, −50, −70, or −80° C.

The cartridge may be designed so that it is compatible with the instrument that performs the subsequent analysis, such as a flow cytometer. The cartridge may be transported to the cytometry instrument, which is at a remote location in one embodiment, after the cell samples are contacted with the reagents. The cartridge may have alignment markings or structure to allow the cartridge to be inserted into the cytometer for immediate processing.

One embodiment of the present invention is a device that includes multiple chambers to hold the cell or blood sample and to all the cells to be in contact with the appropriate reagents. Those reagents may include a small molecule, protein, lipid inhibitor, agonist of signal transduction, antagonist of signal transduction, a stimulant/modulator/potentiator, buffer or other cell fixative, and other reagents for the process outlined above relating to flow cytometry. The reagents may include a permeabilizing agent, a fixative agent, an agent that prevents the secretion of proteins from the endoplasmic reticulum or golgi apparatus, or a stain, for example and be stored in the chambers prior to the introduction of the cells or may be added into the chambers after the cell sample or blood is added, using a separate attachment. The reagents may be present in a lyophilized state or may be in a liquid state in a variety of concentrations from dilute to concentrated. They may be added using automated equipment, such as an automated syringe.

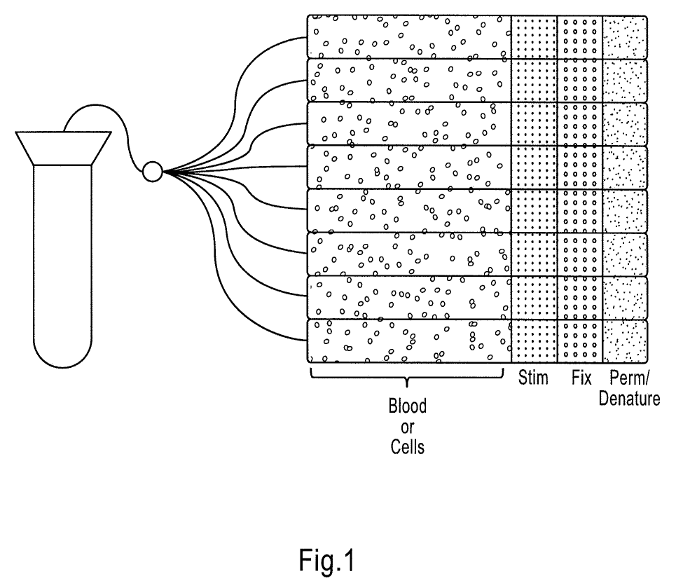

As shown in FIG. 1, an embodiment of the present invention includes a manifold which may be fluidly connected to a cell collection device, such as a blood draw tube, to chambers in a cartridge which are used to contact the cells with the reagents. The manifold may be constructed of rigid or flexible material. Rigid materials can include glass, metals, such as stainless steel, or hard plastics. Flexible materials include plastic tubing, for example. The manifold draws fluid samples from the cell collection device and distributes the fluid to all, or a number of specific chambers through a flow line. Chambers may have one or more compartments wherein different reagents are added or stored or different reactions take place. Seals may be used between chambers or compartments within chambers and may be reversible so that fluids, samples, reagents, etc. may flow between compartments. There may optionally be different subcompartments within the compartments. They may be released in any appropriate fashion together or individually. The reagents may be placed in a linear order to release the ones closest to the cell sample.

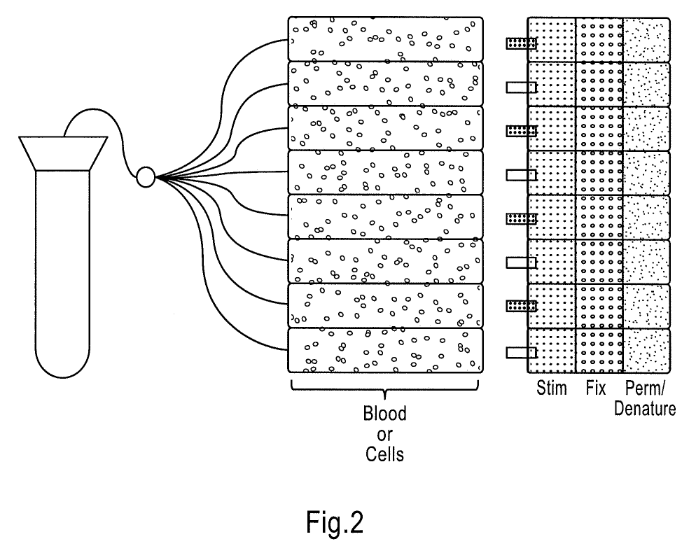

As illustrated in FIG. 2, the cartridge may have a separate cassette, which can contain the stimulants for alignment with the chambers in much the same fashion as shown above for FIG. 1. The cassette may have pins, clamps, attachments, connectors, depressed or raised regions that align with the chambers. The reagents may be present in either solid or liquid form for the embodiments in all Figures. Also, the cassette, manifold, cartridge and all fluid lines are preferably fluid tight and may use seals and o-rings. Other compartments or lines may be attached to the chambers for additional reagents. Another embodiment of the invention can use a blood collection device, a manifold, a flow line, and chambers for a microtiter like plate.

In one embodiment of the present invention, the manifold may be constructed to contain multiple fluid lines from one line which is connected to the initial sample collection device. The initial line may be fluidly connected to all other lines or the initial line may be connected to other lines in series. A valve may be inserted between the initial line and other lines to control flow and volume. A pump may be used to direct the appropriate volume to each chamber.

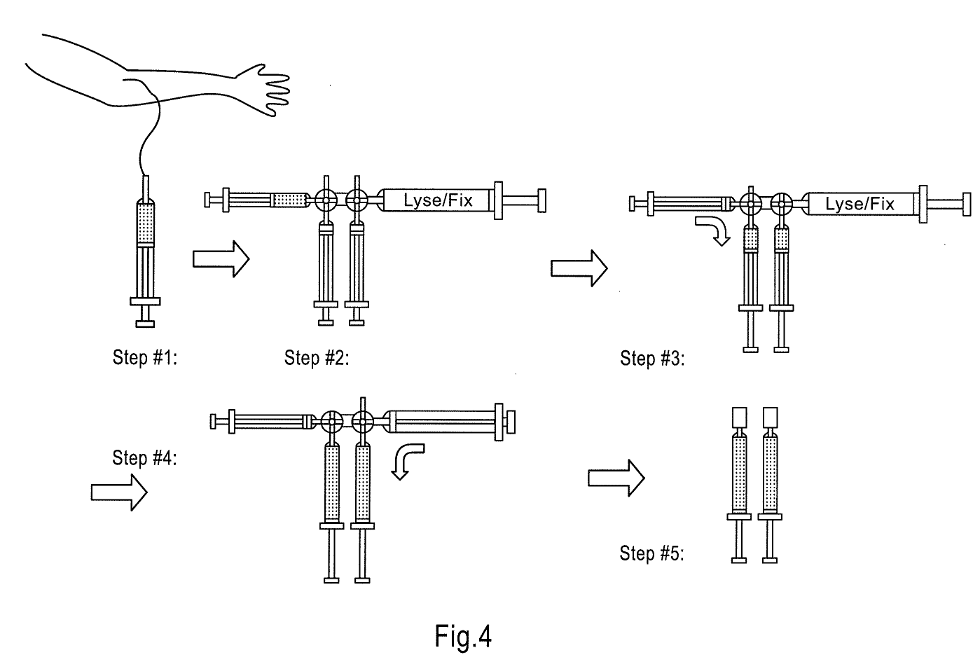

Another embodiment of the present invention is shown in FIG. 4. It is an embodiment that is not complex and shows how blood may be drawn and directly attached to a manifold for sample processing. One example process is outlined in which the whole blood sample may be inserted into syringes holding stimulators, then a lyse/buffer may be inserted to fix the cells. Then the samples may be further processed.

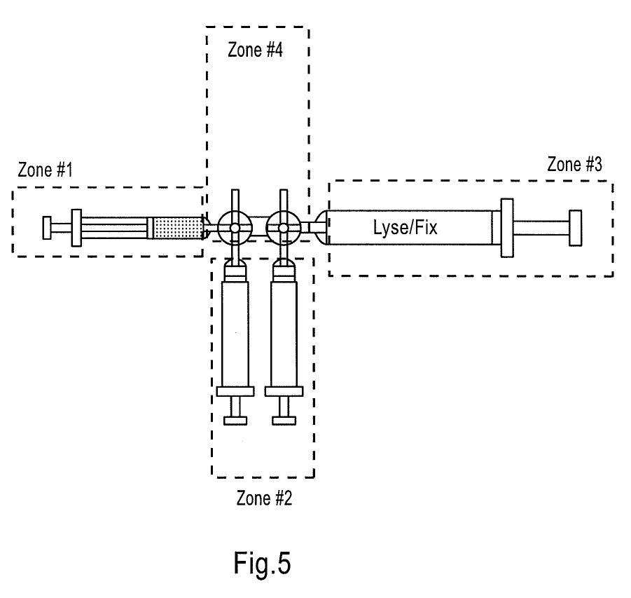

FIG. 5 also shows some additional details surrounding the embodiment of FIG. 4. FIG. 5 depicts 4 discrete zones. Zone one is the blood collection device (i.e. tube or syringe). Zone two contains prefilled syringe(s), tube(s), container(s), or plate(s) that may contain various agents, such as proteins, lipids, antibodies, small molecules which potentiate or inhibit cellular signal transduction pathways. Zone three is a container with a fixative that simultaneously lyses red blood cells and fixes while blood cells while preserving phosphorylated eptitopes for later measurement using flow cytometry. Zone four contains a manifold containing either manually or electronically controlled valves to control the direction of fluid movement between zones. The valves may be controlled manually with, for example, stopcocks or electronically timed with a computer.

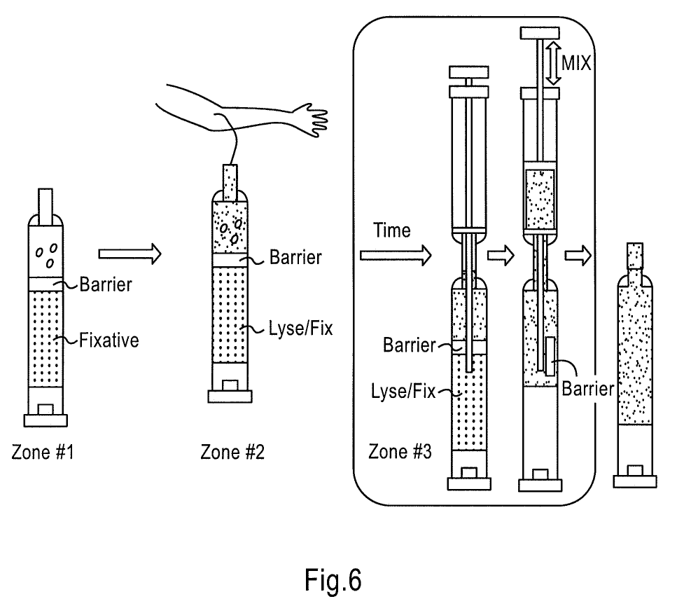

Another embodiment of the present invention is shown in FIG. 6. FIG. 6 shows three discrete zones. In zone one is syringe containing a fixative on one side of a barrier and a modifier of signal transduction on the other side of the barrier. The barrier can be made, for example of a gel or silicon. The blood once collected would be contained in the top of the syringe with the modifier of signal transduction. The blood sample could then be incubated with the modifier of signal transduction for a sufficient amount of time so as to either effectively antagonize or potentiate the desired signal transduction pathway. Zone two, is similar to zone one except that zone two contains a combination fixative/lysis solution for the lysis of red blood cells. Zone three shows the introduction of another programmable time controlled syringe pump that when activated pierces the barrier layer between the fixative or lyse/fix solution and the blood sample thereby mixing the two solutions. The syringe can then be use to facilitate the mixing of the fixative or lyse/fix solution with the solution of blood and signal transduction modifier.

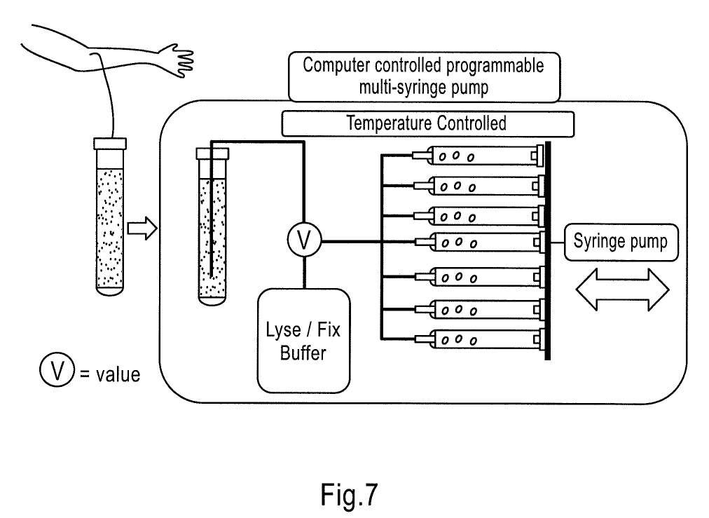

FIG. 7 shows another embodiment of the present invention in which the samples are subject to higher parallel processing. The process may be automated to achieve higher consistency, efficiency and speed.

The system of the present invention is capable of distributing specified and accurate liquid volumes to the selected chambers. Appropriate electronic and manual valve devices may be used to ensure that liquid is placed in the correct chambers. For example, the liquid handling devices recited above can be used to measure liquid volumes. Automated syringes, may be used or other metered pumps. Valves may include the standard physical devices that are present on laboratory equipment that rotate with manual stopcocks or electronic valves, or may include the types of valves or liquid shunting devices or methods that are present with lab-on-a-chip type devices where liquid volumes can be transferred to selected chambers using positive or negative pressures or other valving. See for example U.S. Pat. No. 6,830,936 and U.S. Publication Nos. 2007157973; 20060258019; 20020079008; 20060134599; and 20020125139. See also Seiler, K. et al., “Electroosmotic Pumping and Valveless Control of Fluid Flow Within a Manifold of Capillaries on a Glass Chip,” Anal. Chem. 66:3485-3491 (1994); Cheng X, Irimia, et. al., Practical label-free CD4+ T cell counting of HIV-infected subjects: A Microchip approach. Lab on a Chip 2007; 10:1039; Demirci U, Toner M., Direct etch method for microfluidic channel and nanoheight post-fabrication by picoliter droplets, Applied Physics Letters 2006; 88 (5), 053117; and Irimia D, Geba D, Toner M., Universal microfluidic gradient generator, Analytical Chemistry 2006; 78: 3472-3477.

In one embodiment of the invention, the manifold is fluidly connected to the chambers. The chambers may be grouped in one device such as a cartridge, block, plate, or otherwise as a unit (hereinafter called a “cartridge” for convenience). In one embodiment, the number of chambers in the cartridge is any whole number from 2 to 1,000, preferably, the number of chambers is at least 3, 4, 5, 6, 7, 8, 9, or 10 and no more than 750, 500, 400, 300, 200, or 100. The cartridge may be a custom design or may be commercially available, such as microtiter plates having 94 384, or 1536 wells, for example.

The blood or other cell sample may be moved into the chambers by gravity, positive or negative pressure. The chambers may have a vacuum, a pump or pressurized gas source may be used to push the fluid into the chambers and the like. Automated equipment for this task has been described above. An example of programmable syringe pump is made, for example, by Harvard Apparatus (Holliston, Mass.).

The cartridge may be reusable or disposable and may be composed of a wide range of material, either biological, nonbiological, organic, inorganic, or a combination of any of the above. The cartridge may have any convenient three dimensional shape, such as a rectangular block, cube, sphere, circle, etc. The interior of the chamber is preferably smooth or flat, but may take on a variety of alternative surface configurations. For example, it may contain raised or depressed regions which may serve various decorative or functional purposes, such as mixing. The cartridge may be made of glass, fused quartz, polymerized Langmuir Blodgett film, functionalized glass, Si, Ge, GaAs, GaP, SiO2, SiN4, modified silicon, or any one of a wide variety of gels or polymers such as (poly)tetrafluoroethylene, (poly)vinylidenedifluoride, polystyrene, polycarbonate, ABS plastic, polyvinylchloride, polyethylene, products sold under the trademarks TEFLON™ and KALREZ™ and the like, among others or combinations thereof. Other materials with which the cartridge can be composed of will be readily apparent to those skilled in the art upon review of this disclosure.

Internal and external surfaces on the solid cartridge will usually, though not always, be composed of the same material as the cartridge itself. Thus, the surface that contacts the sample or air may be composed of any of a wide variety of materials, for example, polymers, plastics, resins, polysaccharides, silica or silica-based materials, carbon, metals, inorganic glasses, membranes, or any of the above-listed materials.

The resulting cartridge will have a variety of uses including, for example, screening cell samples for responses to one or more stimulators, modulators or potentiators, either together or individually. The one or more modulators are shown in the patents, applications and articles shown above. The compounds in the cells (such as phosphorylated cell signaling proteins) that respond to the modulators can be labeled with a detectable marker, such as a marker that can be detected with, for example, a flow cytometer or a mass spectrometer. Other detectors or instruments may also be employed. See the references above for information on instruments, including flow cytometers and the associated processes for their use in the present invention.

The cartridge may be made of an array of standard blood collection tubes held together with appropriate clamping or framework. Variations of arrays of blood tubes may be used in the cartridge. An example of a blood tube is made, for example by The Sarstedt Group (Numbrecht, Germany). The cartridge can be manufactured from injection molded plastic. Injection molding enables the casings to be formed inexpensively. If it is desirable to have multiple pieces, then the pieces can be mated with substantially complementary pieces to form a finished assembly. Also, assembling the package from two or more parts simplifies the construction of various features, such as the internal channels for introducing fluids into the chamber. As a result, the packages may be manufactured at a relatively low cost.

Preferably, the chambers are sufficiently large enough to accommodate an appropriate volume of fluid. For example, in one embodiment, the chamber can contain at least 0.001, 0.01, 0.1, 0.5, 1, 2, 3, 4, 5, 6, 7, 8, 9, 10 or more microliters, or no more than 100, 75, 50, 25 or 10 microliters. In another embodiment the chamber can contain at least 0.1, 0.5, 1, 2, 3, 4, 5, 6, 7, 8, 9, 10 or more milliliters (mls) of fluid, or no more than 100, 75, 50, 25 or 10 mls.

In some embodiments, it is important that there are a sufficient number of cells for the analysis, such as flow analysis. A larger volume of blood could be necessary if there was no concentration of cells and less volume would be required if there was such a step. Filters to concentrate cells are referred to above. See U.S. Ser. No. 09/790,630.

If the cells of interest were concentrated, then smaller volume cartridges may be used, such as the lab-on-a-chip devices, referred to above. For cartridges that are used in these devices a typical cartridge can be about 0.5 to 3″ wide, 0.5 to 4″ long, and 0.1 to 1″ high or more preferably 1 to 2″ wide, 1 to 3″ long, and 0.5 to 1″ high. Preferably, the cartridge is of sufficient size to accommodate identification labels, RFIDs, or bar codes in addition to the chambers. Cartridges in which the chambers have a volume of between 2 and 10 mls, may be between 2 to 10″ wide, 2 to 12″ long, and 1 to 3″ high, or more preferably 5 and 9″ wide, 3 and 9″ long, and 1 to 2″ high.

The chamber may have any conceivable size, shape, or orientation. Preferably, the chamber has a volume sufficient to allow the cells to contact the one or more reagents, such as a modulator, and then mixed with other reagents, like a fixative, permeabilizing reagent or a stain. In one embodiment, the chamber may be at least 0.2″ wide, 0.2″ long, and 0.2″ deep. For small volume chambers the volume can be in the microliter scale, such as the volumes described above.

In one embodiment, the cartridge can be open on two or more ends or access points. Preferably, the cell sample can be inserted through an inlet, port or opening. In one embodiment, there is only one opening to insert the cell sample because the appropriate reagents are pre-deposited in the chamber to await the cells. In another embodiment of the present invention, the cartridge is constructed to be opened to insert the sample, then it is closed to incubate or otherwise mix the sample plus the reagents. Another embodiment has another access point to add the reagents to the cell sample, such as blood, after the blood is inserted into the chamber. The reagents can be added individually or in one operation. A separate device, as a cassette, may be attached to the cartridge to add the reagents. The use of a cassette having areas that align to the chambers of the cartridge may further enable high-throughput processing of the samples in lieu of the slower process of individual addition. Alignment marks and structure can be added to facilitate an appropriate match between the cartridge and devices with the reagents. Alternatively, the reagents may be added to the chambers in the cartridge through the manifold opening once the manifold is removed. Automated dispensers, such as those described below involving jetting or pumping, are shown below.

Selected fluids can be introduced into and out of the chamber via inlets. In some embodiments, the inlets are located at opposite ends of the chamber. This configuration improves fluid circulation and regulation of bubble formation in the chamber for mixing. The bubbles agitate the fluid, increasing the contact between the one or more modulators and cell. Other methods to mix the sample are known in the art. In one embodiment, the inlets are located at the top and bottom end of the chamber when the package is oriented vertically, such as at the opposite corners of the chamber. Locating the inlet at the highest and lowest positions in the chamber facilitates the removal of bubbles from the chamber if desired. Internal structure can also be used in the chamber to facilitate mixing. Such structure can be pegs, posts, or other physical structure that can disrupt the fluid flow to enhance mixing.

In one embodiment of the invention, the cartridge has chambers with an inlet for the cell sample and an inlet for the reagents. The reagents are added by attaching a structure with or without its own chamber, which may align the reagents with the chambers for contact with the cells. The structure may also have a connection to add further reagents according to a particular timed schedule as shown in the patent applications identified above.

Once the first reagent is added, the cells and the fluid in the chamber may be subject to agitation to improve contact of the first, or the other reagents with the cells. See U.S. Pat. No. 6,399,365 for examples of agitation systems. The agitation can involve external shaking or internal fluid circulation. During any of these procedures, the manifold may be removed and the chamber openings may be covered with a seal to prevent leakage and evaporation. Ports or inlet may be used to add further reagents or the cover may be removed periodically for addition. The cover (or any other seal) may be attached via clips, clamps, screws, adhesives, and other fasteners.

One embodiment of the invention includes a cartridge, which could include a microtiter type plate, and a manifold which deposits cell samples into each well of the microtiter plate. The plate may be conventional and commercially available, or it may be a custom design. The number of wells may be 96, 384, 1536 or other standard sizes. The volume may be as stated above, at least 1, 2, 3, 4, 5, 6, or 7 or more mls of fluid, typically blood. Microtiter plates may be obtained from commercial suppliers such as those listed above. The microtiter plate may have predeposited reagents as mentioned above.

In another embodiment, one or more reagents may be delivered by a dispenser capable of aliquotting fluids to individual wells along an X-Y axis. See U.S. Pat. No. 6,121,048. Specified reactants may be delivered to certain wells which can be identified by encoded information on the cartridge (bar code, RFID, magnetic coding, etc.) controlled by a processor such as a computer. The reactants can be delivered to precise locations by, for example, piezoelectric pump, pipettes, micropipettes, electrophoretic pumps, or mechanisms adapted from ink-jet printing technology. The appropriate volume of reagents will contribute to the device that is selected.

It should be noted that the even distribution of fluid flow through the chamber prevents dead zones from occurring in the chamber. For example, the even distribution of fluid through the chamber substantially prevents fluid from becoming substantially quiescent at certain locations.

One embodiment of the cartridge can include a seal at any opening of the chamber. It may be a septum composed of rubber, teflon/rubber laminate, or other sealing material. The septum may be of the type commonly used to seal and reseal vessels when a needle is inserted into the septum for addition/removal of fluids. The septums, when seated in the depressions, extend slightly above surface, which in some embodiments is about 0.01″ to 0.05″.

Optionally, a temperature control mechanism such as a heater, a cooler, or a combination thereof can be disposed next to the cartridge. The temperature control mechanism can be any suitable thermally controlled element such as a resistive element, a temperature controlled block or mass, thermoelectric modules, or the like. The temperature control mechanism transfers heat or cold via conduction to the cartridge, which transfers heat or cold to fluid in the chamber. Alternatively, the temperature control mechanism sinks heat away from, for example, fluid in the chamber. The temperature control mechanism maintains a selected temperature in the chamber. The temperature control mechanism also includes a temperature detection device such as a thermocouple which provides signals corresponding to temperature readings. A controller receives the signals corresponding to the temperature readings, and adjusts power output to the temperature control mechanism to maintain the selected temperature.

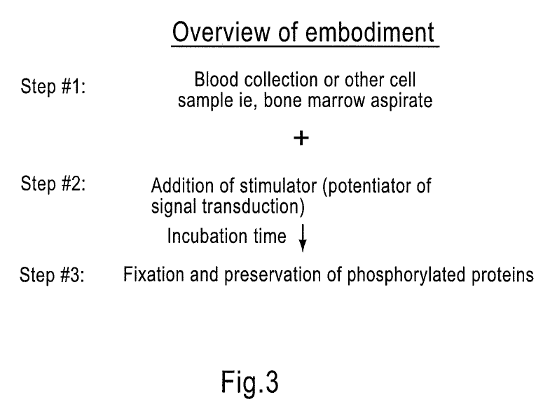

There are various reagents that can be added to the chambers before or after addition of the cell sample. They can be added in solid (powder, lyophilized, etc.) or liquid form. As shown in the references listed above, one preferred process of the present invention is phosphoflow cytometry. In that process, cells are contacted with one or more modulators or potentiators to stimulate the cells, then the cells are fixed with a buffer containing reagent, and then the cells may be permeabilized to allow reagent access across the cell membrane, then additional reagents are contacted with the cells to “stain” particular proteins within the cell. Thereafter, the cells are analyzed on a flow cytometer to detect the presence or absence of the stains. See U.S. Ser. Nos. 10/193,462; 11/655,785; 11/655,789; 10/346,620; 11/655,821; 10/898,734; and 11/338,957 which are all incorporated by reference in their entireties. See FIG. 3 for one example of an embodiment of the present invention.

The instant invention also makes use of cells that have been “potentiated.” In contrast to “activation,” a “potentiated” state refers the state of a cell after exposure to a potentiator which then can be activated as the case may be. As described in detail below, potentiators exert their effect on signaling cascades by directly or indirectly impacting the ability of an activatible protein to switch between activation isoforms.

Potentiators include chemical and biological entities, and physical or environmental stimuli. Potentiators can act extracellularly or intracellularly. Chemical and biological potentiators include growth factors, cytokines, neurotransmitters, adhesion molecules, hormones, small molecules, inorganic compounds, polynucleotides, antibodies, natural compounds, lectins, lactones, chemotherapeutic agents, biological response modifiers, carbohydrate, proteases and free radicals. Physical and environmental stimuli include electromagnetic or particulate radiation, redox potential and pH, the presence or absences of nutrients, changes in temperature, oxygen partial pressure, ion concentrations and oxidative stress. Potentiators can be endogenous or exogenous and may produce different effects depending on the concentration and duration of exposure to the single cells or whether they are used in combination or sequentially with other potentiators.

The potentiators include ligands for cell surface receptors (for example the potentiators include IL-2, EGF, GMCSF, TNFα, Toll-like receptor ligands such as lipopolysaccaride, double stranded RNA, poly I:C, bacterial lipoprotiens, flagellin, unmethylated CpG DNA, etc). Examples of such receptor elements include compounds or events that will activate hormone receptors, cytokine receptors (ILI-a, IL-b, IL-2, IL-3, IL-4, IL-5, IL-6, IL-7, IL-8, IL-9, IL-10. IL-12, IL-15, IL-18, IL-21,), chemokine receptors (CCR5, CCR7, CCR1-10, CCR20, CXCR4, RANTES, MIP-1α, MIP-1β, IP-10, MCP-1, IL-8) steroid receptors (estrogen receptors, thyroid hormone receptors, androgen receptors glucocorticoid receptors, etc), adhesion receptors (VCAM (vascular cell adhesion molecule), ICAM (intracellular adehsion molecule), integrin receptors, selectins, etc) and growth factor receptors (PDGF-R (platelet derived growth factor receptor), EGF-R (epidermal growth factor receptor), VEGF-R (vascular endothelial growth factor), uPAR (urokinase plasminogen activator receptor), ACHR (acetylcholine receptor), IgE-R (immunoglobulin E receptor), integrin receptors (β1, β2, β3, β4, β5, β6, α1, α2, α3, α4, α5, α6), MAC-1 (β2 and cd11b), αVβ33, opioid receptors (mu and kappa), FC receptors, serotonin receptors (5-HT, 5-HT6, 5-HT7), β-adrenergic receptors, insulin receptor, leptin receptor, statin receptors, FAS receptor, BAFF receptor, FLT3 receptor, GMCSF receptor, and fibronectin receptor. Specifically contemplated are potentiators that specifically relate to any of the particular receptors noted above, such as IL-2 for the IL-2 receptor, and VEGF-R for the VEGF receptor, for example.

Commercially available potentiators include: Phorbol 12-Myristate 13-Acetate; Ionomycin, Thapsigargin, LPS, Poly I:C, unmethylated CpG DNA, CD40L, SCF, IGF-1, IL-6, IL-10, Etoposide, IL-3, SDF-1a/CXCL12, HydroxyUrea, Z-VAD-FMK Caspase Inhibitor, G-CSF, Erythropoetin (EPO), SDF-1B/CXCL12, IL-27, M-CSF, GM-CSF, FLT-3 Ligand, VEGF, and TRAIL.

Other potentiators are selected from the group consisting of H₂O₂, siRNA, miRNA, calcium, Cantharidin, (−)-p-Bromotetramisole, Microcystin LR, Sodium Orthovanadate, Sodium Pervanadate, Vanadyl sulfate, Sodium oxodiperoxo(1,10-phenanthroline)vanadate, bis(maltolato)oxovanadium(IV), Sodium Molybdate, Sodium Perm olybdate, Sodium Tartrate, Imidazole, Sodium Fluoride, β-Glycerophosphate, Sodium Pyrophosphate Decahydrate, Calyculin A, Discodermia calyx, bpV(phen), mpV(pic), DMHV, Cypermethrin, Dephostatin, Okadaic Acid, NIPP-1, N-(9, 10-Dioxo-9, 10-dihydro-phenanthren-2-yl)-2,2-dimethyl-propionamide, α-Bromo-4-hydroxyacetophenone, 4-Hydroxyphenacyl Br, α-Bromo-4-methoxyacetophenone, 4-Methoxyphenacyl Br, α-Bromo-4-(carboxymethoxy)acetophenone, 4-(Carboxymethoxy)phenacyl Br, and bis(4-Trifluoromethylsulfonamidophenyl)-1,4-diisopropylbenzene, phenyarsine oxide, Pyrrolidine Dithiocarbamate, and Aluminium fluoride.

In certain embodiments, the assay and screening methods of the invention include fixing the cells. This step is performed to preserve or “freeze” a cell in a certain state, preferably so that an accurate representation of the structure of the cell is maintained. For example, it is often desirable to maintain the cell's original size and shape, to minimize loss of cellular materials, and/or to retain the reactivity and/or status of its intracellular constituents (for example, the cell's phosphorylation level). Cells may be fixed by any of a variety of suitable chemical and physical methods. Preferably, such a method is compatible with multi-well plate format assays. Methods of cell fixation typically rely on crosslinking and/or rapid dehydration agents, such as formaldehyde, paraformaldehyde, glutaraldehyde, acetic acid, picric acid, methanol, ethanol, and acetone. Other suitable fixation agents are, Preferably, one or more fixing agents are added to cells contained in the well of an assay plate. Cells are preferably incubated in the presence of the fixing agent at a certain temperature (for example at room temperature, i.e., between 18° C. and 25° C.) and for a certain period of time (for example between 5 and 10 minutes). Fixation of cells in whole blood preferably would hypotonically lyse the red blood cells while simultaneously fixing and preserving the white blood cells. Excess fixing agent may be removed after centrifugation by aspiration of the supernatant.

Wash buffers can be used to “fix” a cell after stimulation with a potentiator. Wash buffers are know in the art, see for example, U.S. Pat. No. 7,326,577 and U.S. Pub. No. 2006/0141549, which are hereby incorporated by reference in its entireties. One exemplary fixation buffer suitable for whole blood samples is BD™ Phosflow Lyse/Fix Buffer (BD Biosciences, Franklin Lakes, N.J.).

Current fixatives revolve primarily around alcohol and formaldehyde/paraformaldehyde, Jacobberger, J W, Flow Cytometric Analysis of Intracellular Protein Epitopes. Immunophenotyping 2000; 361-409. The fixative described by Connelly (Pizzolo, G, et al. Detection of membrane and intracellular antigens by flow cytometry following ORTHO PermeaFix fixation. Leukemia. 1994 April; 8(4):672-6) is the best single step fixative and permeation agent discovered to date (see Metso, T, et al., Identification of intracellular markers in induced sputum and bronchoalveolar lavage samples in patients with respiratory disorders and healthy persons. Respir Med. 2002 November; 96(11):918-26) stating that “Best results were obtained using a commercial reagent Ortho PermeaFix (OPF) for flow cytometry”). It is called Ortho PERMEAFIX™, although that product has been replaced with a new product called PERMIFLOW™ (INVIRION, INC.™ MI). OPF and its variants are well described in U.S. Pat. No. 5,422,277 and U.S. Pat. No. 5,597,688. Preferred fixatives comprised 0.756%-0.85% formaldehyde, 25.4-30 mM DNBS, 6.9-6.92% DMSO and 0.086-0.095% TWEEN 20 detergent, although many variations are described.

OPF fixation is asserted to have “maintained the morphology of lymphoid cells with minimal cellular distortion and scatter changes, and only slightly modified cell surface immunoreactivity.” Pizzolo G, et al. Detection of membrane and intracellular antigens by flow cytometry following ORTHO PermeaFix fixation. Leukemia. 1994 April; 8(4):672-6. Fixative has been used for detection of both surface and intracellular antigens, Francis C & Connelly M C, Rapid single-step method for flow cytometric detection of surface and intracellular antigens using whole blood, Cytometry. 1996 Sep. 1; 25(1):58-70; U.S. Pat. No. 5,422,277 and U.S. Pat. No. 5,597,688. OPF can be compatible with DNA staining, S & Cheta N, Permeafix: a useful tool to detect antigens and DNA in flow cytometry, Rom J Intern Med. 1997 January-December; 35(1-4):133-5.

Once fixed, the cells can be pelleted and resuspended in methanol to permeabilize the cell membrane, although other methods of permeabilization such as, for example, TWEEN™ 20, are also compatible with the instant invention. Cells can be stored at this point or combined with labeled binding elements and analyzed.

Permeabilization is performed to facilitate access to cellular cytoplasm or intracellular molecules, components or structures of a cell. In particular, permeabilization may allow an agent (such as a phospho-selective antibody) to enter into a cell and reach a concentration within the cell that is greater than that which would normally penetrate into the cell in the absence of such permeabilizing treatment.

Permeabilization of the cells may be performed by any suitable method (see, for example, C. A. Goncalves et al., Neurochem. Res. 2000, 25: 885-894). These methods include, but are not limited to, exposure to a detergent (such as CHAPS, cholic acid, deoxycholic acid, digitonin, n-dodecyl-β-D-maltoside, lauryl sulfate, glycodeoxycholic acid, n-lauroylsarcosine, saponin, and triton X-100) or to an organic alcohol (such as methanol and ethanol). Other permeabilizing methods comprise the use of certain peptides or toxins that render membranes permeable (see, for example, O. Aguilera et al., FEBS Lett. 1999, 462: 273-277; and A. Bussing et al., Cytometry, 1999, 37: 133-139). Permeabilization may also be performed by addition of an organic alcohol to the cells. Selection of an appropriate permeabilizing agent and optimization of the incubation conditions and time can easily be performed by one of ordinary skill in the art. Cells may be permeabilized in the presence of 90% methanol and incubated on ice for 30 minutes. Following this treatment, the assay plate may be stored at −20° C. for up to one month before being analyzed. Permeabilization can occur concurrently with the fixation step. With for example, BD™ Cytofix/Cytoperm (BD Biosciences, Franklin Lakes, N.J.).

Furthermore, it may be necessary to block secretion of proteins from cells. Agents that block intracellular protein transport are well known in the art and include, for example brefeldin A and monensin. However, any agent that is effective in blocking protein secretion from cells may be used.

In general, there are a variety of ways to detect the activation state of a particular protein (i.e. activatible element). In one embodiment, labeled binding elements (“BEs”) are used, which bind specifically to one isoform of the protein. Alternatively the state of the activatible protein is used for the readout; for example, in the case of cell surface receptors with signaling domains, the activity (or lack thereof) of the signaling domain can be assayed directly. For example, the two isoforms may be no activity (negative signal) versus kinase activity (measured using chromogenic substrates).

By “binding element,” “BE,” and grammatical equivalents thereof, is meant any molecule, e.g., nucleic acids, small organic molecules, and proteins which are capable of detecting one isoform of an element over another.

In a preferred embodiment, the protein BE is an antibody. In a particularly preferred embodiment, the protein BE is an activation state-specific antibody. The antigenicity of an activated isoform of an activatable protein is distinguishable from the antigenicity of a non-activated isoform of an activatable protein or from the antigenicity of an isoform in a different activation state. For example, an activated isoform of a protein may possess an epitope that is absent in an on-activated isoform or vice vera. Additionally, moeities, such as phosphate groups, may be covalently added to the proteins, or the structure of the protein may be altered by cleavage or another conformational change with causes the protein to present the same sequence in an antigenically distinguishable way. Accordingly, the methods and compositions of the present invention may be used to detect any particular element isoform in a sample that is antigenically detectable and antigenically distinguishable from other isoforms of the activatible element that are present in the sample. For example, the activation state-specific antibodies of the present invention can be used in the present methods to identify distinct signaling cascades of a subset or subpopulation of complex cell populations; and the ordering of protein activation (e.g., kinase activation) in potential signaling hierarchies.

By “antibody” herein is meant a protein consisting of one or more polypeptides substantially encoded by all or part of the recognized immunoglobulin genes. The recognized immunoglobulin genes, for example in humans, include the kappa (k), lambda (I), and heavy chain genetic loci, which together comprise the myriad variable region genes, and the constant region genes mu (u), delta (d), gamma (g), sigma (e), and alpha (a) which encode the IgM, IgD, IgG, IgE, and IgA isotypes respectively. Antibody herein is meant to include full length antibodies and antibody fragments, and may refer to a natural antibody from any organism, an engineered antibody, or an antibody generated recombinantly for experimental, therapeutic, or other purposes as further defined below. The term “antibody” includes antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. Particularly preferred are full length antibodies that comprise Fc variants as described herein. The term “antibody” comprises monoclonal and polyclonal antibodies. Antibodies can be antagonists, agonists, neutralizing, inhibitory, or stimulatory.

Many antibodies, many of which are commercially available (for example, see Cell Signaling Technology's and Becton Dickinson's catalogues, the contents which are incorporated herein by reference) have been produced which specifically bind to the phosphorylated isoform of a protein but do not specifically bind to a non-phosphorylated isoform of a protein. Many such antibodies have been produced for the study of signal transducing proteins that are reversibly phosphorylated. In particular, many such antibodies have been produced which specifically bind to phosphorylated, activated isoforms of protein kinases and are sometimes referred to herein as kinase activation state antibodies or grammatical equivalents thereof. Examples of phospho-specific antibodies include antibodies to cell cycle proteins such as cyclin dependent kinases (cdk) such as, for example, p-Cdk (Thr14/Thyr15), pCdk2 (Thr 160), or phospho p27 (Ser 10), phospho p27 (Thr 187) or phospho p21 (Thr 145), or proteins involved in tumor suppression or apoptosis such as, for example, p-Bad (Ser 112), pBcl-2 (Ser 87), pBID (6D3), p-caspase-6-p10 (Ser 257), p-p53 (mSer20) (Thr 18) (Thr 377), p-PTEN (Ser380) or p-Rb (Thr 356) or transcriptional regulators such as p-c-myc (Ser 373), p-c-Jun (Ser-73) or p-c-Fos (34E4) or steroid receptors such as, for example, pAR (4H24), p-ERa (Ser 118), p-ERb (Ser 87) or p-PR (1154) or lymphocyte signaling proteins such as p-CD133 (Thr 266), p-CD3zeta (C415.9A), pCD45 (Ser 940) or pCD88 (32-G1). A more exhaustive list of these antibodies and others useful in the current invention can be found for example in the catalog of Santa Cruz Biotechnology, Inc. Particularly preferred antibodies for use in the present invention include: phospho-AKT Ser473 monoclonal anti-4E2, phospho-p44/42 MAP kinase (Thr202/Tyr204) monoclonal antibody, phospho-TYK2 (Tyr1054/1055) antibody, phospho-p38 MAP kinase (Thr180/Tyr182) monoclonal antibody 28B 10, phospho-PKC-PAN substrate antibody, phospho-PKA-substrate, phospho-SAPK/JNK (Thr183/Tyr185) G9 monoclonal antibody, phospho-tyrosine monoclonal antibody (P-tyr-100), p44/42 MAPK, p38 MAPK, JNK/SAPK, and phospho-AKT-Thr308.

The methods and compositions of the instant invention provide BEs comprising a label or tag. By label is meant a molecule that can be directly (i.e., a primary label) or indirectly (i.e., a secondary label) detected; for example a label can be visualized and/or measured or otherwise identified so that its presence or absence can be known. A compound can be directly or indirectly conjugated to a label which provides a detectable signal, e.g. radioisotopes, fluorescers, enzymes, antibodies, particles such as magnetic particles, chemiluminescers, specific binding molecules, or molecules that can be detected by mass spectroscopy etc. Specific binding molecules include pairs, such as biotin and streptavidin, digoxin and antidigoxin etc. Preferred labels include, but are not limited to, optical fluorescent and chromogenic dyes including labels, label enzymes radioisotopes, and quantum dots. Most preferred labels include for example, PE (phycoerythrobilin), FITC (fluroscein isothiocyanate), APC (allophycocyanin), GFP (green florescent protein), PerCP (peridinin chlorophyll protein) and CFSE (carboxyfluoroscein).

One embodiment of the invention includes the addition of one or more reagents to the chamber prior to addition of the cell sample. The reagents may be in solid or liquid form and may include the potentiator and/or the fixative. Another embodiment of the invention would involve additionally depositing the permeability and/or staining agent within the chamber prior to the addition of the cell sample.

In many embodiments, there is an appropriate timing with reagent addition. Since some of the reagents need to be added after allowing the potentiator to act, then a mechanism for timed release would be useful. Methods for timed release are known in the art and include physical structure or chemical formulations that are designed to release a reagent at a particular time after an event like adding the sample. Physical devices include an enclosure like an ampoule that may be broken to release a reagent. Other physical devices include structure having a porous nature that will release a reagent over time, such as a porous membrane. Chemical formulations that allow the timed release of reagents include micelles, liposomes, cleavable linkers attached to the reagent and/or another molecule, and the like. See U.S. Pat. Nos. 6,004,572; 5,079,005, and 5023080.

In one embodiment, the reagents are added to the chambers prior to the addition of the cell sample. This embodiment allows the cartridge to be preloaded and makes the process simpler for the user, who may be at the point of blood draw or care. Such person may be less skilled in the particular assay and may have various other duties to perform in addition to sample collection and preparation. It would be advantageous to simplify the process with fewer steps.

In another embodiment, the reagents are added after the cell samples are added to the chambers. This embodiment would require manual addition of reagents or a mechanism to automatically add the reagents to all chambers. Automatic addition at one time would allow flexibility to design the stim package individually to the particular samples or disease.

However, it would require some local processing by the user. This embodiment preferably employs a device that could be simply attached to the cartridge to deliver the reagents. After delivery, it could be removed and the cartridge sealed from the environment or it could remain attached and the reagents contacted with the cell samples.

The reagent(s), such as the potentiator may be added as a solid (it could be added as a liquid then lyophilized prior to cell sample addition) or liquid prior to or after the cell sample is added to the cartridge. The cell sample could be added via a manifold that would be fluidly connected to the cell sample source, such as whole blood. The blood could be transported to the cartridge/microtiter plate via the manifold and deposited into wells of the plate. The reagents could be placed in contact with the cells, either by being in the wells already or by being added thereafter.

In one embodiment of the invention, a system to provide notice to the operators of the cytometer lab that the samples are in transit may be conducted over the internet. U.S. Patent Publication 20050009078 discusses the use of internet ordering systems useful in the present invention.

In some embodiments, the present inventions provide commercially feasible devices for mixing a plurality of cells with reagents that are used for flow cytometry. It is to be understood that the above description is intended to be illustrative and not restrictive. Many embodiments will be apparent to those skilled in the art upon reviewing the above description. Merely as an example, the package may be molded or machined from a single piece of material instead of two. Also, other asymmetrical designs may be employed to orient the package onto the detection systems.

The scope of the invention should, therefore, be determined not with reference to the above description, but instead should be determined with reference to the appended claims along with their full scope of equivalents.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments.

Also, the functions of several elements may, in alternative embodiments, be carried out by fewer, or a single, element. Similarly, in some embodiments, any functional element may perform fewer, or different, operations than those described with respect to the illustrated embodiment. Also, functional elements shown as distinct for purposes of illustration may be incorporated within other functional elements in a particular implementation. Certain functional elements, files, data structures, and so on may be described in the illustrated embodiments as located in system memory of a particular computer or instrument. In other embodiments, however, they may be located on, or distributed across, computer systems, instruments, or other platforms that are co-located and/or remote from each other. For example, any one or more of data files or data structures described as co-located on and “local” to a server or other computer may be located in a computer system or systems remote from the server. In addition, it will be understood by those skilled in the relevant art that control and data flows between and among functional elements and various data structures may vary in many ways from the control and data flows described above or in documents incorporated by reference herein. More particularly, intermediary functional elements may direct control or data flows, and the functions of various elements may be combined, divided, or otherwise rearranged to allow parallel processing or for other reasons. Also, intermediate data structures or files may be used and various described data structures or files may be combined or otherwise arranged. Numerous other embodiments, and modifications thereof, are contemplated as falling within the scope of the present invention as defined by appended claims and equivalents thereto.

### EXAMPLES

### Example 1

### See FIGS. 4 and 5

1. Remove phosphoflow blood collection apparatus from refrigerator and remove from packaging.
2. Allow the apparatus to sit at room temperature for least 15 minutes so that buffers will equilibrate to room temperature.
3. 3 ml of blood is collected via syringe (BD Eclipse™ Hypodermic Needle) attached to either a 3 cc or Scc syringe following manufactures instructions.
   3.1. Remove needle from syringe
4. Secure syringe to manifold as shown in FIG. 4 step #2.
   4.1. Twist the syringe firmly to lock it to the manifold
5. 3-way stopcock valve should be positioned to allow blood to flow to the receiving syringes (See Step #3, FIG. 4)
6. Inject approximately 1 ml of blood into receiving syringes by pressing blood collection syringe.
   6.1. Mix the blood by inverting the apparatus 5 times.
   6.2. Start Timer
7. Let apparatus sit for the stated time listed on the apparatus. Different stimuli may require different stimulation time. A typical incubation time is 15 minutes at room temperature.
8. Position stopcock valve as shown in Step #4 in FIG. 4.
9. Inject Lyse/Fix buffer into receiving syringes
   9.1. Mix the blood+Lyse/Fix buffer by inverting the apparatus 5 times.
10. Remove receiving syringes containing the fixed blood samples from the apparatus
   10.1. Cap Tightly
11. Store at −20° C. to −80° C. for up to 1 month.

### Example 2

### See FIG. 6

1. Remove phosphoflow blood collection vacuum tubes from refrigerator to and remove from packaging.
2. Allow the tubes to sit at room temperature for least 15 minutes so that buffers will equilibrate to room temperature.
3. Collect blood (see Zone #2 in FIG. 6)
4. Mix by inverting tube 5 times.
5. Place tubes into the programmable syringe pump and set pump to the program # listed on the blood tube.
   5.1. After a specified incubation time the pump will activate and piece the barrier inside the blood tube (see Zone# 3 in FIG. 6).
6. After syringe pump has finished mixing the Lyse/fix and blood together, remove tube and cap tightly.
7. Store at −20° C. to −80° C. for up to 1 month.

### Example 3

### See FIG. 7

1. Collect blood 8-9 mls of blood in a 10 cc heparin containing Vacutainer tube (BD Biosciences).
2. Invert 5 times to mix the blood and heparin together
3. Load computer controlled programmable phosphoflow device with a cassette of syringes containing various stimuli.
4. Make sure Lyse/Fix reservoir is filled.
5. Select appropriate program number.
6. Place Vacutainer under the aspiration port as shown in FIG. 7, so that the long needle pierces the rubber stopper.
7. Press “Start”.
8. The syringe pump will engage and draw up the blood into a series of syringes in the cassette. Depending on the program number selected, the pump will pause for a set amount of time to allow the stimulus to activate the cells.
9. At a specified time (depending on the program selected), the valve “V” (FIG. 7) will activate to allow the pump to draw up Lyse/fix buffer into the syringes.
10. When program ends, remove the cassette of syringes.
   10.1. Cap each syringe.
11. Store at −20° C. to −80° C. for up to 1 month.

### Example 4

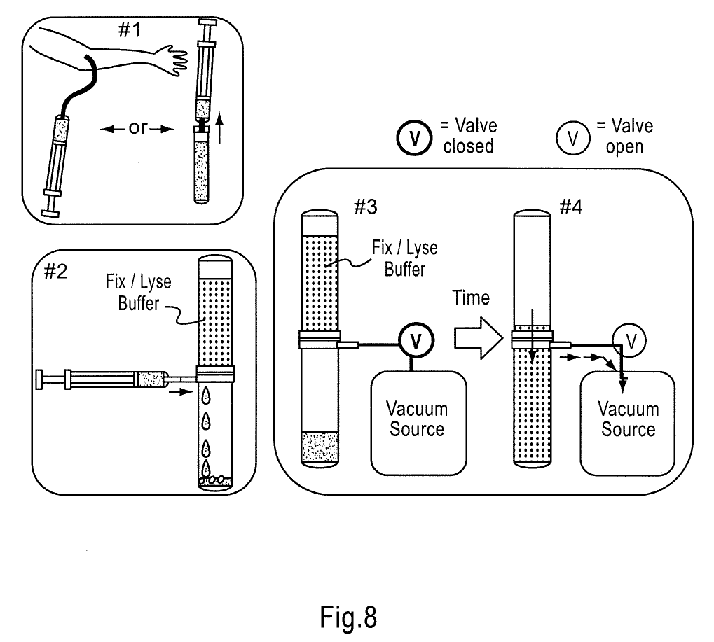

### See FIG. 8

### Materials

Device to draw blood, such as Safety multifly set made by Sarstedt. Includes needle, butterfly, tube and connector to syringe. Alternatively, using a syringe, one could draw blood out of a vacutainer (BD Biosciences) already containing a blood sample.

Tubes that can be divided by valve or membrane, such as Steriflips, which contains a 50 ml tube and a porous membrane on the top and a nozzle on the side to draw negative pressure. Tubes may be prefilled with a modulator in any physical form (lyophilized, frozen, liquid, powder);

Corresponding tubes attached to the top of the Steriflip (Falcon).

Liquid solution of lyse and fix buffer. Lyse is for RBCs and fix is to fix the internal protein environment of cell. Solution is in the Falcon tubes which can be attached to the Steriflips;

Vacuum source, syringe, pump, or house vacuum

### Method

Draw blood into syringe;

Disconnect needle from syringe;

Connect syringe to first tube via injection port;

Inject blood into first tube, having the modulator, through a nozzle. First tube has filter/valve attached on top. Swirl to mix blood/modulator and incubate at the appropriate temperature for a prescribed amount of time;

Fluidly attach second tube to first tube through valve on top. These tubes can be pre-attached and sent in a kit. Draw vacuum through nozzle of first tube to cause the buffer from the second tube to flow into the first tube and to contact the cells;

Invert several times to mix cells and buffer and cap tube. Discard other equipment and ship to lab for testing.

Can use different hardware to separate reaction from reagents and use vacuum to move fluids around. First tube may have two chambers, one with stim, one without. Valve is inside, blood and buffer are drawn into both chambers via vacuum through nozzle attached to reaction chamber.

### Kit

Two tubes are attached with valve in between. First one has port/nozzle on one side of the valve and that tube contains the stim. The other contains the buffer. Both are prefilled. Syringe has heparin or EDTA for anticoagulant effect. Simply inject blood into first tube, draw vacuum and buffer is introduced. Discard tube with buffer and ship to lab. Include shipping label with kit.

Another embodiment comprises two chambers, one for reactions and one for reagent storage. A port for sample introduction and for vacuum connection is included. The chamber for reactions may comprise the stim, either preloaded (one use) or connected to a port that can be used to introduce the stim or other reagents.

What is claimed is:

1. A system for preparing cells for analysis, comprising:
at least one cartridge containing a plurality of chambers;
a manifold fluidly connected to the said plurality of chambers;
at least one fluid line to deliver a plurality of cells to the chambers; and
a plurality of reagents fluidly connected to the said plurality of chambers

2. A method for preparing cells for flow cytometery analysis, comprising:
obtaining a plurality of cells from an individual;
directing the said plurality of cells through a manifold into a plurality of chambers;
contacting the cells in the said chambers with one or more reagents; and
analyzing the cells.

3. The system of claim 1, wherein the plurality of reagents comprises at least one, a series, or combination of modulators and is present in the plurality of chambers prior to addition of the plurality of cells.

4. The system of claim 1, wherein the plurality of reagents comprises a modulator and is present in the plurality of chambers after addition of the plurality of cells.

5. The method of claim 2, further comprising contacting the cells with a stain in the chambers and detecting a stain on the plurality of cells.

6. The system of claim 1, wherein the cells are present in whole blood.

7. The method of claim 2, further comprising drawing the cells into the chambers using a vacuum.

8. The system of claim 6, wherein a vent is added to the chamber holding the whole blood.

9. The system of claim 1, wherein the volume of the chamber is between 0.1 microliters and 10 mls.

10. The method of claim 2, wherein the volume of the chamber is between 0.1 microliters and 10 mls.

11. The system of claim 1, wherein the chambers are closed end tubes that are held together in a framework.

12. The system of claim 1, wherein the chambers are in a microtiter plate.

13. The system of claim 1, wherein the chambers are more than one cartridge.

14. The system of claim 1, wherein the chambers are in a lab-on-a-chip and a device is used to concentrate cells of interest.

15. The system of claim 1, wherein the chambers are in a microtiter plate having 94, 384 or 1536 wells.

16. The system of claim 1, wherein a chamber has an identifier, such as a bar code, a fluorescent color coding bar code, an RFID, or DNA bar code.

17. The method of claim 2 wherein the chambers are agitated using bubbles or physical agitation.

18. The system of claim 1, wherein a filter is added between the whole blood and chamber.

19. The system of claim 1, wherein the modulator is added with an attachable device which is fluidly connected to a plurality of chambers.

20. The system of claim 1, wherein the attachable device can be designed to customize which modulator is inserted into which chamber.

21. A cartridge for preparing cells for flow cytometry, comprising:
a housing including a plurality of fluid chambers constructed and arranged to retain fluid containing cells, said housing including a bar code;
the housing is fluidly connected to a source containing cells;
a manifold fluidly connected to the housing;
cell stimulating reagents; and
time release cell fixing buffers.

22. A cell analysis system comprising:
a fluid container comprising a plurality of cells;
a plurality of chambers;
a fluid delivery line to connect the fluid container to the plurality of chambers;
an automated device for programmed delivery of liquid or solid reagents to the plurality of chambers; and
a flow cytometer.

23. A cell analysis system of claim 22, wherein an automated syringe is used to deliver reagents.

24. A computer system for controlling fluid flow into a number of chambers in the system of claim 1, comprising software stored on storage medium.
